(19) United States
(12) Patent Application Publication
(10) Pub. No.: US 2025/0134472 A1
(43) Pub. Date: May 1, 2025

(54) EAR-WEARABLE DEVICES AND SYSTEMS WITH ORTHOSTATIC INTOLERANCE CONDITION SCREENING FEATURES

(71) Applicant: Starkey Laboratories, Inc., Eden Prairie, MN (US)

(72) Inventors: Roy Rozenman, Mishmar David (IL); Nitzan Bornstein, Tel Aviv (IL)

(21) Appl. No.: 18/683,554

(22) PCT Filed: Aug. 16, 2022
(86) PCT No.: PCT/US2022/040473

§ 371 Date: Feb. 14, 2024

Related U.S. Application Data
(60) Provisional application No. 63/234,071, filed on Aug. 17, 2021.

(51) Int. Cl. A61B 5/00; A61B 5/0205; A61B 5/021; A61B 5/024; A61B 5/026; A61B 5/11

(52) U.S. Cl. CPC A61B 5/7282; A61B 5/02055; A61B 5/486; A61B 5/6817; A61B 5/7285; A61B 5/746; A61B 5/747; A61B 5/021; A61B 5/02433; A61B 5/0261; A61B 5/1116; A61B 5/1118; A61B 2562/0219

(57) ABSTRACT

Embodiments herein relate to ear-wearable devices and systems that can be used to screen for orthostatic intolerance conditions. In an embodiment, an ear-wearable device can be included having a control circuit, a microphone, an electroacoustic transducer, and a sensor package. The sensor package can include a motion sensor and an optical sensor. The ear-wearable device can be configured to process signals from the motion sensor to detect a postural transition of a device wearer to a standing position, trigger operation of the optical sensor, and process signals from the optical sensor to screen for an orthostatic intolerance condition. Other embodiments are also included herein.

This application is being filed as a PCT International Patent application on Aug. 16, 2022, in the name of Starkey Laboratories, Inc., a U.S. national corporation, applicant for the designation of all countries, and Roy Rozenman, a Citizen of Israel, and Nitzan Bornstein, a Citizen of Israel, inventors for the designation of all countries, and claims priority to U.S. Provisional Patent Application No. 63/234,071 filed Aug. 17, 2021, the contents of which are herein incorporated by reference in its entirety.

### FIELD

Embodiments herein relate to ear-wearable devices and systems that can be used to screen for orthostatic intolerance conditions.

### BACKGROUND

Orthostatic intolerance conditions include those wherein movement from sitting or lying to an upright posture causes symptomatic arterial hypotension (orthostatic hypotension). Orthostatic intolerance syndromes can occur when the autonomic nervous system is impaired and fails to respond to the challenges imposed by the upright posture. Orthostatic intolerance syndromes can also occur because of volume depletion rendering the body unable to maintain blood pressure due to decreased circulating volume.

Drugs may cause orthostatic intolerance conditions as a side effect. For example, diuretics and vasodilators cause central volume depletion. Further, tricyclic antidepressants, phenothiazines, antihistamines, and MAO-inhibitors can directly affect the central nervous system. Alcohol consumption can directly affect the central nervous system and cause central volume depletion.

Disease states may also lead to orthostatic intolerance conditions. For example, primary degenerative diseases of the central nervous system can cause orthostatic intolerance conditions. In addition, other diseases such as diabetes mellitus, kidney or liver failure can result in damage to the autonomic nervous system leading to orthostatic intolerance conditions.

Postural orthostatic tachycardia syndrome (POTS) is a specific form of orthostatic intolerance in which the impairment of the autonomic nervous system to respond to an upright position is partially counteracted by a compensatory tachycardia.

### SUMMARY

Embodiments herein relate to ear-wearable devices and systems that can be used to screen for orthostatic intolerance conditions. In a first aspect, an ear-wearable device can be included having a control circuit, a microphone, an electroacoustic transducer, and a sensor package. The sensor package can include a motion sensor and an optical sensor. The ear-wearable device can be configured to process signals from the motion sensor to detect a postural transition of a device wearer to a standing position, trigger operation of the optical sensor, and process signals from the optical sensor to screen for an orthostatic intolerance condition.

In a second aspect, in addition to one or more of the preceding or following aspects, or in the alternative to some aspects, the orthostatic intolerance condition can include a condition selected from the group consisting of postural orthostatic tachycardia syndrome and orthostatic hypotension.

In a third aspect, in addition to one or more of the preceding or following aspects, or in the alternative to some aspects, the motion sensor can include at least one sensor selected from the group consisting of an accelerometer and a gyroscope.

In a fourth aspect, in addition to one or more of the preceding or following aspects, or in the alternative to some aspects, the ear-wearable device can be configured to process signals from the optical sensor to detect inter-beat interval changes.

In a fifth aspect, in addition to one or more of the preceding or following aspects, or in the alternative to some aspects, the ear-wearable device can be configured to identify decreased variability in inter-beat interval changes or another statistical measure as indicative of an orthostatic intolerance condition.

In a sixth aspect, in addition to one or more of the preceding or following aspects, or in the alternative to some aspects, the ear-wearable device can be configured to process signals from the optical sensor to detect pulse rate changes.

In a seventh aspect, in addition to one or more of the preceding or following aspects, or in the alternative to some aspects, the ear-wearable device can be configured to process signals from the optical sensor to detect pulse rate changes exceeding a threshold value.

In an eighth aspect, in addition to one or more of the preceding or following aspects, or in the alternative to some aspects, the ear-wearable device can be configured to process signals from the optical sensor to determine a blood flow morphology.

In a ninth aspect, in addition to one or more of the preceding or following aspects, or in the alternative to some aspects, the ear-wearable device can be configured to process signals from the optical sensor to determine a blood flow morphology consistent with reduced systolic blood pressure.

In a tenth aspect, in addition to one or more of the preceding or following aspects, or in the alternative to some aspects, the ear-wearable device can be configured to process signals from the optical sensor to determine a magnitude of change in an AC portion of the signals.

In an eleventh aspect, in addition to one or more of the preceding or following aspects, or in the alternative to some aspects, the ear-wearable device can be configured to process signals from the optical sensor to determine a recovery rate in the AC portion of the signals to normal levels.

In a twelfth aspect, in addition to one or more of the preceding or following aspects, or in the alternative to some aspects, the ear-wearable device can be configured to process signals from the optical sensor to determine a magnitude and duration of change in the AC portion of the signals.

In a thirteenth aspect, in addition to one or more of the preceding or following aspects, or in the alternative to some aspects, the optical sensor can include a light emitter.

In a fourteenth aspect, in addition to one or more of the preceding or following aspects, or in the alternative to some aspects, the light emitter emits light at a near-infrared wavelength.

In a fifteenth aspect, in addition to one or more of the preceding or following aspects, or in the alternative to some aspects, wherein triggering operation of the optical sensor includes turning on the light emitter.

In a sixteenth aspect, in addition to one or more of the preceding or following aspects, or in the alternative to some aspects, the ear-wearable device can be configured to prompt the device wearer to stand.

In a seventeenth aspect, in addition to one or more of the preceding or following aspects, or in the alternative to some aspects, the ear-wearable device can be configured to issue a command to an accessory device to prompt the device wearer to stand.

In an eighteenth aspect, in addition to one or more of the preceding or following aspects, or in the alternative to some aspects, the ear-wearable device can be configured to query the device wearer regarding their condition after they assume a standing position.

In a nineteenth aspect, in addition to one or more of the preceding or following aspects, or in the alternative to some aspects, the ear-wearable device can be configured to evaluate a reaction speed of the device wearer after they assume a standing position.

In a twentieth aspect, in addition to one or more of the preceding or following aspects, or in the alternative to some aspects, the ear-wearable device can be configured to send an alert to a care provider if a possible orthostatic intolerance condition is detected.

In a twenty-first aspect, in addition to one or more of the preceding or following aspects, or in the alternative to some aspects, the ear-wearable device can be configured to process signals from the motion sensor to detect postural sway after the device wearer assumes a standing position.

In a twenty-second aspect, in addition to one or more of the preceding or following aspects, or in the alternative to some aspects, the ear-wearable device can be configured to issue a warning to the device wearer if postural sway crossing a threshold value is detected.

In a twenty-third aspect, in addition to one or more of the preceding or following aspects, or in the alternative to some aspects, the ear-wearable device can be configured to process signals from the motion sensor to determine a starting posture prior to transition of the device wearer to a standing position.

In a twenty-fourth aspect, in addition to one or more of the preceding or following aspects, or in the alternative to some aspects, the ear-wearable device can be configured to trigger operation of the optical sensor after detection of the postural transition of the device wearer to the standing position when the postural transition was preceded by lying or sitting for at least a threshold amount of time.

In a twenty-fifth aspect, in addition to one or more of the preceding or following aspects, or in the alternative to some aspects, the ear-wearable device can be configured to warn the device wearer if an orthostatic intolerance condition is detected.

In a twenty-sixth aspect, in addition to one or more of the preceding or following aspects, or in the alternative to some aspects, the ear-wearable device can be configured to warn the device wearer after detecting a sedentary period of the device wearer if orthostatic intolerance conditions were previously detected.

In a twenty-seventh aspect, in addition to one or more of the preceding or following aspects, or in the alternative to some aspects, the ear-wearable device can be configured to warn the device wearer after detecting the beginning of a transition to a standing posture if orthostatic intolerance conditions were previously detected.

In a twenty-eighth aspect, in addition to one or more of the preceding or following aspects, or in the alternative to some aspects, the ear-wearable device can be configured to adjust a fall risk threshold if an orthostatic intolerance condition is detected.

In a twenty-ninth aspect, in addition to one or more of the preceding or following aspects, or in the alternative to some aspects, the ear-wearable device can be configured to prompt the device wearer to take a precautionary action if an orthostatic intolerance condition is detected.

In a thirtieth aspect, in addition to one or more of the preceding or following aspects, or in the alternative to some aspects, the precautionary action can include sitting back down.

In a thirty-first aspect, in addition to one or more of the preceding or following aspects, or in the alternative to some aspects, the precautionary action can include using an assistive device.

In a thirty-second aspect, in addition to one or more of the preceding or following aspects, or in the alternative to some aspects, the precautionary action can include performing an exercise.

In a thirty-third aspect, in addition to one or more of the preceding or following aspects, or in the alternative to some aspects, the ear-wearable device can be configured to prompt the device wearer to drink water if an orthostatic intolerance condition is detected.

In a thirty-fourth aspect, in addition to one or more of the preceding or following aspects, or in the alternative to some aspects, the ear-wearable device can be configured to provide stimulation to the device wearer when a postural transition to a standing position can be detected to mitigate the effects of an orthostatic intolerance condition.

In a thirty-fifth aspect, in addition to one or more of the preceding or following aspects, or in the alternative to some aspects, the sensor package can include a temperature sensor.

In a thirty-sixth aspect, in addition to one or more of the preceding or following aspects, or in the alternative to some aspects, the ear-wearable device can be configured to evaluate a signal from the temperature sensor while screening for an orthostatic intolerance condition.

In a thirty-seventh aspect, in addition to one or more of the preceding or following aspects, or in the alternative to some aspects, the ear-wearable device can be configured to evaluate a signal from the temperature sensor and suspend screening for the orthostatic intolerance condition if the temperature sensor indicates a temperature falling outside of a predetermined range.

In a thirty-eighth aspect, in addition to one or more of the preceding or following aspects, or in the alternative to some aspects, the ear-wearable device can be configured to process signals from the motion sensor over a time period prior to transition of the device wearer to a standing position to characterize an activity level of the device wearer.

In a thirty-ninth aspect, in addition to one or more of the preceding or following aspects, or in the alternative to some aspects, the ear-wearable device can be configured to evaluate the activity level and suspend screening for the orthostatic intolerance condition if the activity level crosses a threshold value.

In a fortieth aspect, in addition to one or more of the preceding or following aspects, or in the alternative to some aspects, the ear-wearable device can be configured to set a threshold value for detection of an orthostatic intolerance condition based at least in part on at least one factor of the device wearer selected from age, known diagnosis, BMI, footstep speed, known gait instability, known history of orthostatic intolerance conditions, and trends thereof.

In a forty-first aspect, in addition to one or more of the preceding or following aspects, or in the alternative to some aspects, the ear-wearable device can be configured to set a threshold value for detection of an orthostatic intolerance condition based at least in part on known alcohol use, cardiovascular disease status, allergies, blood disorders, and prescribed medications.

In a forty-second aspect, a method for screening for orthostatic intolerance conditions using an ear-wearable system can be included. The method can include processing signals from a motion sensor to detect a postural transition of a device wearer to a standing position, triggering operation of the optical sensor, and processing signals from the optical sensor to screen for an orthostatic intolerance condition.

In a forty-third aspect, in addition to one or more of the preceding or following aspects, or in the alternative to some aspects, the orthostatic intolerance condition can include a condition selected from the group consisting of postural orthostatic tachycardia syndrome and orthostatic hypotension.

In a forty-fourth aspect, in addition to one or more of the preceding or following aspects, or in the alternative to some aspects, the method can further include processing signals from the optical sensor to detect inter-beat interval changes.

In a forty-fifth aspect, in addition to one or more of the preceding or following aspects, or in the alternative to some aspects, the method can further include processing signals from the optical sensor to detect pulse rate changes.

In a forty-sixth aspect, in addition to one or more of the preceding or following aspects, or in the alternative to some aspects, the method can further include processing signals from the optical sensor to detect pulse rate changes exceeding a threshold value.

In a forty-seventh aspect, in addition to one or more of the preceding or following aspects, or in the alternative to some aspects, the method can further include processing signals from the optical sensor to determine a blood flow morphology.

In a forty-eighth aspect, in addition to one or more of the preceding or following aspects, or in the alternative to some aspects, the method can further include processing signals from the optical sensor to determine a blood flow morphology consistent with reduced systolic blood pressure.

In a forty-ninth aspect, in addition to one or more of the preceding or following aspects, or in the alternative to some aspects, the method can further include processing signals from the optical sensor to determine a magnitude of change in the AC portion of the signals.

In a fiftieth aspect, in addition to one or more of the preceding or following aspects, or in the alternative to some aspects, the method can further include processing signals from the optical sensor to determine a recovery rate in the AC portion of the signals to normal levels.

In a fifty-first aspect, in addition to one or more of the preceding or following aspects, or in the alternative to some aspects, the method can further include processing signals from the optical sensor to determine a magnitude and duration of change in the AC portion of the signals.

In a fifty-second aspect, in addition to one or more of the preceding or following aspects, or in the alternative to some aspects, the method can further include issuing a command to an accessory device to prompt the device wearer to stand.

In a fifty-third aspect, in addition to one or more of the preceding or following aspects, or in the alternative to some aspects, the method can further include querying the device wearer regarding their condition after they assume a standing position. In a fifty-fourth aspect, in addition to one or more of the preceding or following aspects, or in the alternative to some aspects, the method can further include evaluating a reaction speed of the device wearer after they assume a standing position.

In a fifty-fifth aspect, in addition to one or more of the preceding or following aspects, or in the alternative to some aspects, the method can further include sending an alert to a care provider if a possible orthostatic intolerance condition is detected.

In a fifty-sixth aspect, in addition to one or more of the preceding or following aspects, or in the alternative to some aspects, the method can further include detecting postural sway after the device wearer assumes a standing position.

In a fifty-seventh aspect, in addition to one or more of the preceding or following aspects, or in the alternative to some aspects, the method can further include processing signals from the motion sensor to determine a starting posture prior to transition of the device wearer to a standing position.

In a fifty-eighth aspect, in addition to one or more of the preceding or following aspects, or in the alternative to some aspects, the method can further include triggering operation of the optical sensor after detection of the postural transition of the device wearer to the standing position when the postural transition was preceded by lying or sitting for at least a threshold amount of time.

In a fifty-ninth aspect, in addition to one or more of the preceding or following aspects, or in the alternative to some aspects, the method can further include warning the device wearer if an orthostatic intolerance condition is detected.

In a sixtieth aspect, in addition to one or more of the preceding or following aspects, or in the alternative to some aspects, the method can further include warning the device wearer after detecting a sedentary period of the device wearer if orthostatic intolerance conditions were previously detected.

In a sixty-first aspect, in addition to one or more of the preceding or following aspects, or in the alternative to some aspects, the method can further include warning the device wearer after detecting the beginning of a transition to a standing posture if orthostatic intolerance conditions were previously detected.

In a sixty-second aspect, in addition to one or more of the preceding or following aspects, or in the alternative to some aspects, the method can further include adjusting a fall risk threshold if an orthostatic intolerance condition is detected.

In a sixty-third aspect, in addition to one or more of the preceding or following aspects, or in the alternative to some aspects, the method can further include prompting the device wearer to take a precautionary action if an orthostatic intolerance condition is detected.

In a sixty-fourth aspect, in addition to one or more of the preceding or following aspects, or in the alternative to some aspects, the method can further include prompting the device wearer to drink water if an orthostatic intolerance condition is detected.

In a sixty-fifth aspect, in addition to one or more of the preceding or following aspects, or in the alternative to some aspects, the method can further include providing stimulation to the device wearer when a postural transition to a standing position can be detected to mitigate the effects of an orthostatic intolerance condition.

In a sixty-sixth aspect, in addition to one or more of the preceding or following aspects, or in the alternative to some aspects, the method can further include evaluating a signal from a temperature sensor while screening for an orthostatic intolerance condition.

In a sixty-seventh aspect, in addition to one or more of the preceding or following aspects, or in the alternative to some aspects, the method can further include evaluating a signal from a temperature sensor and suspend screening for the orthostatic intolerance condition if the temperature sensor indicates a temperature falling outside of a predetermined range.

In a sixty-eighth aspect, in addition to one or more of the preceding or following aspects, or in the alternative to some aspects, the method can further include processing signals from the motion sensor over a time period prior to transition of the device wearer to a standing position to characterize an activity level of the device wearer.

In a sixty-ninth aspect, in addition to one or more of the preceding or following aspects, or in the alternative to some aspects, the method can further include setting a threshold value for detection of an orthostatic intolerance condition based at least in part on at least one factor of the device wearer selected from age, known diagnosis, BMI, footstep speed, known gait instability, known history of orthostatic intolerance conditions, and trends thereof.

In a seventieth aspect, in addition to one or more of the preceding or following aspects, or in the alternative to some aspects, the method can further include setting a threshold value for detection of an orthostatic intolerance condition based at least in part on known alcohol use, cardiovascular disease status, allergies, blood disorders, and prescribed medications.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of an ear-wearable device in accordance with various embodiments herein.

FIG. 2 is a schematic view of an ear-wearable device within an ear in accordance with various embodiments herein.

FIG. 3 is a schematic view of a device wearer rising from a chair in accordance with various embodiments herein.

FIG. 4 is a flowchart of operations in accordance with various embodiments herein.

FIG. 5 is an exemplary view of an optical sensor signal in accordance with various embodiments herein.

FIG. 6 is a schematic view of the amplitude of the AC portion of an optical sensor signal in accordance with various embodiments herein.

FIG. 7 is a schematic view of a device wearer in a standing posture in accordance with various embodiments herein.

FIG. 8 is a schematic view of an accessory device in accordance with various embodiments herein.

FIG. 9 is a schematic view of a system in accordance with various embodiments herein.

FIG. 10 is a schematic view of components of an ear-wearable device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

There is substantial value in screening for orthostatic intolerance conditions including orthostatic hypotension and/or postural orthostatic tachycardia syndrome (POTS). For example, if in individual is identified as possibly having an orthostatic intolerance condition then a medical professional can be alerted and can investigate the possible causes and/or consider possible interventions to address the same. In addition, detecting changes in orthostatic intolerance of an individual can provide a medical professional with insight into whether an underlying health condition is improving or worsening. Further, screening for orthostatic intolerance conditions can allow for actions to be taken to reduce the individual's risk of falling and getting injured. However, current diagnostic techniques to identify orthostatic intolerance conditions can generally only be performed in a clinical setting.

Embodiments of ear-wearable devices and systems herein can be used to screen for orthostatic intolerance conditions. Ear-wearable devices herein are uniquely capable of, and valuable for, screening for orthostatic intolerance conditions because such devices, including those used as hearing assistance devices, are typically worn all the time (or nearly all the time) by device wearers. This means that screening can be done conveniently in a natural setting. In addition, screening can be performed much more often to allow changes in orthostatic intolerance to be more quickly and accurately recognized. Further, in some embodiments, screening can be conducted automatically such as when the device or system detects that the device wearer transitions from a lying or sitting posture to a standing posture in the normal course of their activity without bothering the device wearer to take specific actions.

In an embodiment, an ear-wearable device is included having a control circuit, a microphone, an electroacoustic transducer, and a sensor package. The sensor package can include a motion sensor and an optical sensor. The ear-wearable device can be configured to process signals from the motion sensor to detect a postural transition of a device wearer to a standing position, trigger operation of the optical sensor, and process signals from the optical sensor to screen for an orthostatic intolerance condition.

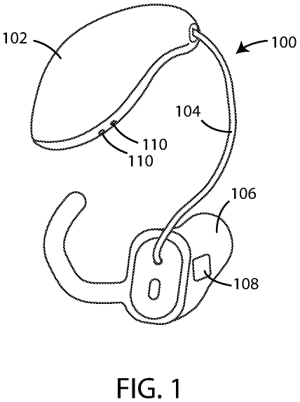

Referring now to FIG. 1, a schematic view of an ear-wearable device 100 is shown in accordance with various embodiments herein. The ear-wearable device 100 includes a housing 102 in which various components of the device can be housed. In this example, the ear-wearable device 100 also includes electrical contacts 110, which can be used for recharging an internal battery. However, some types of ear-wearable devices herein may lack a battery compartment, such as a device with a rechargeable battery. The ear-wearable device 100 also includes a cable 104 which connects to a receiver 106. The ear-wearable device 100 also includes sensor 108, which could be any of the sensors described herein.

In various embodiments, the ear-wearable device 100 can be configured to process signals from the motion sensor to detect a postural transition of a device wearer to a standing position. The motion sensor (described in greater detail below) can include, for example, an accelerometer and/or a gyroscope. A postural transition to a standing position or posture can be detected by evaluating the motion sensor signals to detect characteristic patterns and/or signatures within the motion sensor signals. Further details of pattern identification as can be applied to signal processing are described in greater detail below.

Operation of the optical sensor can consume significant energy. As such, it can be advantageous to only turn on the optical sensor when it is needed. To detect orthostatic intolerance conditions, it may only be necessary to evaluate the signal of an optical sensor as the device wearer begins to transition to a standing position or posture and/or after the device wearer has transitioned to a standing position or posture. As such, the ear-wearable device 100 can be configured to trigger operation of an optical sensor only after a postural transition to a standing position has been detected. In some embodiments, the ear-wearable device 100 can be configured to trigger operation of an optical sensor only after a postural transition to a standing position has been started and/or completed.

However, in some cases, detecting a postural transition to a standing position may not, by itself, be sufficient to enable accurate measurement properties associated with orthostatic intolerance and therefore detect the same. For example, if an individual was exercising and just momentarily crouched before reassuming a standing position, properties associated with orthostatic intolerance, such as hypotension, may not be present or may only be present to a reduced degree. As such, in various embodiments herein, the device and/or system can determine whether conditions are appropriate to try to evaluate properties associated with orthostatic intolerance. In various embodiments herein, the device and/or system can determine whether certain preconditions have been met before evaluating properties associated with orthostatic intolerance.

For example, in various embodiments, the ear-wearable device 100 can be configured to trigger operation of the optical sensor after detection of the postural transition of the device wearer to the standing position when the postural transition was preceded by lying or sitting for at least a threshold amount of time. The specific amount of time of being seated or lying down or otherwise sedentary can vary, but it should be sufficiently long for the effects of orthostatic intolerance to become evident when the individual transitions to a standing position or posture. For example, the specific amount of time can be about 30 seconds, 1 minute, 2 minutes, 3 minutes, 5 minutes, 7 minutes, 10 minutes, 15 minutes or longer, or an amount of time falling within a range between any of the foregoing.

In some cases, that status of the device wearer may be such that screening for orthostatic intolerance conditions can be difficult or otherwise inaccurate. Under such circumstances, it can be useful to suspend screening activities to prevent spurious results. In some cases, signals from a sensor can be used to detect a status of the device wearer that may not allow for accurate screening for orthostatic intolerance conditions. For example, signals from a temperature sensor can be used for this purpose. In one scenario, the presence of a viral infection may prevent accurate screening for orthostatic intolerance conditions. Further, if an individual has a viral infection they may have an elevated temperature. In another scenario, vigorous exercise may increase prevent accurate screening for orthostatic intolerance conditions. Vigorous exercise may also result in an elevated skin temperature. Thus, in various embodiments, the presence of an elevated temperature may indicate that the present condition of the device wearer is not conducive for screening for orthostatic intolerance conditions.

Therefore, in various embodiments, the ear-wearable device 100 can be configured to evaluate a signal from a temperature sensor before or while screening for an orthostatic intolerance condition. In various embodiments, the ear-wearable device 100 can be configured to evaluate a signal from a temperature sensor and suspend screening for the orthostatic intolerance condition if the temperature sensor indicates a temperature falling outside of a predetermined range. In various embodiments, the ear-wearable device 100 can be configured to evaluate a signal from a temperature sensor and suspend screening for the orthostatic intolerance condition if the temperature sensor indicates a temperature that is above a threshold value. In various embodiments, the ear-wearable device 100 can be configured to evaluate a signal from a temperature sensor and suspend screening for the orthostatic intolerance condition if the temperature sensor indicates a temperature that is below a threshold value.

In some scenarios, it can be difficult to accurately screen for orthostatic intolerance conditions if the device wearer's activity level is too high prior to transitioning to a standing position or posture. For example, if the device wearer was vigorously exercising and then temporarily sat down before standing back up, this may mask normally perceptible characteristics of an orthostatic intolerance condition. Thus, in various embodiments, the ear-wearable device 100 can be configured to process signals from the motion sensor over a time period prior (e.g., a look back period) to a detected transition of the device wearer to a standing position to characterize an activity level of the device wearer. Activity levels can be characterized in various ways. As merely one example, activity levels can be characterized based on a statistical measure of motion sensor signals herein, such as a statistical measure of the magnitude of motion sensor signals herein over a time period. In various embodiments, the ear-wearable device 100 can be configured to evaluate the activity level and suspend screening for the orthostatic intolerance condition if the activity level crosses a threshold value. In various embodiments, the ear-wearable device 100 can be configured to evaluate the activity level and suspend screening for the orthostatic intolerance condition if the activity level exceeds a threshold value.

In various embodiments, the ear-wearable device 100 can be configured to trigger operation of an optical sensor after a postural transition to a standing position has been detected (as the posture transition starts, after it is completed, etc.). In some embodiments, the transition to a standing position can be purely of the device wearer's own volition, such as eventually getting up after sitting in a chair. Such a transition can be detected by the device, such as by evaluating signals from a motion sensor as part of a passive screening approach. However, in other embodiments, the device and/or system can issue an instruction or prompt to the device wearer (audio, video, and/or haptic) to get them to transition to a standing position as part of an active screening approach. For example, the device may recognize that conditions are optimal to screen for an orthostatic intolerance condition (such as following a sufficiently long period of sitting or lying down) and then prompt the user to assume a standing position.

Thus, in various embodiments, the ear-wearable device 100 can be configured to prompt a device wearer to stand. Such an instruction or prompt can be delivered to the device wearer either directly through the ear-wearable device or through an accessory device, or both. In various embodiments, the ear-wearable device 100 can be configured to issue a command to an accessory device to prompt a device wearer to stand.

In various embodiments, the ear-wearable device 100 can be configured to warn the device wearer if an orthostatic intolerance condition is detected. The drop in blood pressure associated with orthostatic intolerance may lead to unsteadiness, lack of coordination, and/or syncope which may lead to falls and/or accidental injury. Thus, in some embodiments, a warning can be provided to the device wearer so that they can exercise appropriate caution.

However, it will be appreciated that in some scenarios a warning provided after an orthostatic intolerance condition has already been detected may be too late to prevent injury. Thus, in various embodiments, the ear-wearable device 100 can be configured to warn a device wearer after detecting a beginning of a transition to a standing posture if orthostatic intolerance conditions were previously detected. Similarly, in various embodiments, the ear-wearable device 100 can be configured to warn a device wearer after detecting a sedentary period of the device wearer if orthostatic intolerance conditions were previously detected. As such, the ear-wearable device can be used to provide reminders to the device wearer to exercise appropriate caution when they stand up based on their past personal history of orthostatic intolerance condition events.

Beyond warnings, various other actions can be taken by the ear-wearable device for individuals for which orthostatic intolerance conditions have been detected. As another example, in various embodiments, the ear-wearable device 100 can be configured to adjust a fall risk threshold of the device if an orthostatic intolerance condition is detected. This can be useful for ear-wearable device that include features to monitor for falls. In various embodiments, the ear-wearable device 100 can be configured to prompt the device wearer to take a precautionary action (such as maintaining contact with a chair or other steadying structure) if an orthostatic intolerance condition is detected, currently or previously.

In various embodiments, the ear-wearable device can be configured to execute various actions to mitigate or ameliorate the effects of an orthostatic intolerance condition. For example, dehydration can lead to blood volume reduction which can, in turn, result in orthostatic intolerance conditions and/or worsening of the same. Thus, in various embodiments, the ear-wearable device 100 can be configured to prompt the device wearer to drink water if an orthostatic intolerance condition is detected.

In various embodiments, the ear-wearable device 100 can be configured to provide stimulation to the device wearer when a postural transition to a standing position is detected to mitigate effects of an orthostatic intolerance condition. In some embodiments, the stimulation provided can be effective to raise the blood pressure of the device wearer to counteract the hypotension that may otherwise occur. For example, the device can provide audio stimulation to the device wearer. In some embodiments, the device can provide queries to the device wearer to provide mental stimulation.

In various embodiments, the ear-wearable device 100 can be configured to set a threshold value for screening of an orthostatic intolerance condition based at least in part on at least one factor of the device wearer selected from age, known diagnosis, BMI, footstep speed, known gait instability, known history of orthostatic intolerance conditions, and trends thereof. For example, for an individual with no risk factors for an orthostatic intolerance condition, the threshold value (in terms of sensor signals referred to elsewhere herein as indicative of an orthostatic intolerance condition) can be increased and/or be relatively high to reduce false positives (increasing specificity but reducing sensitivity). Conversely, for an individual with one or more risk factors for an orthostatic intolerance condition, the threshold value (in terms of sensor signals referred to elsewhere herein as indicative of an orthostatic intolerance condition) can be decreased and/or relatively low to be sure that all possible occurrences of orthostatic intolerance are captured (decreasing specificity but increasing sensitivity). Various other conditions can serve as risk factors for the occurrence of an orthostatic intolerance condition beyond those referenced above. For example, in various embodiments, the ear-wearable device 100 can be configured to set a threshold value for detection of an orthostatic intolerance condition based at least in part on known alcohol use, cardiovascular disease status, allergies, blood disorders, and prescribed medications.

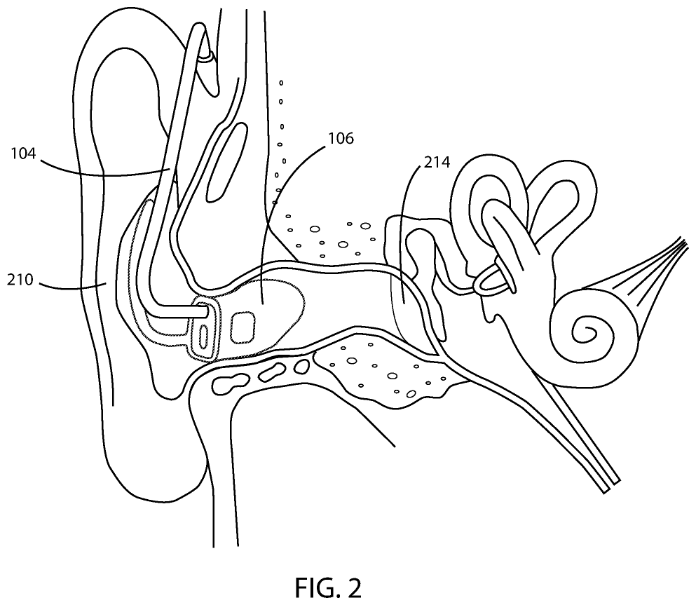

Referring now to FIG. 2, a schematic view of an ear-wearable device 100 within an ear is shown in accordance with various embodiments herein. The ear-wearable device 100 includes a cable 104 and receiver 106. The ear of the device wearer includes a pinna 210, an ear canal 212, and a tympanic membrane 214.

It will be appreciated that while FIGS. 1 and 2 illustrate one type of ear-wearable device consistent with embodiments herein, many other types of ear-wearable devices are also contemplated. The term “ear-wearable device” as used herein shall refer to devices that can aid a person with impaired hearing. The term “ear-wearable device” shall also refer to devices that can produce optimized or processed sound for persons with normal hearing. Ear-wearable devices herein can include hearing assistance devices. Ear-wearable devices herein can include, but are not limited to, behind-the-ear (BTE), in-the ear (ITE), in-the-canal (ITC), invisible-in-canal (IIC), receiver-in-canal (RIC), receiver in-the-ear (RITE) and completely-in-the-canal (CIC) type hearing assistance devices. In some embodiments, the ear-wearable device can be a hearing aid falling under 21 C.F.R. § 801.420. In another example, the ear-wearable device can include one or more Personal Sound Amplification Products (PSAPs). In another example, the ear-wearable device can include one or more cochlear implants, cochlear implant magnets, cochlear implant transducers, and cochlear implant processors. In another example, the hearing assistance device can include one or more “hearable” devices that provide various types of functionality. In other examples, ear-wearable devices can include other types of devices that are wearable in, on, or in the vicinity of the user's ears. In other examples, ear-wearable devices can include other types of devices that are implanted or otherwise osseointegrated with the user's skull; wherein the device is able to facilitate stimulation of the wearer's ears via a bone conduction pathway. In another example, the hearing assistance device can include an auditory brainstem implant, a cranial nerve (e.g., CN VIII) implant, and the like.

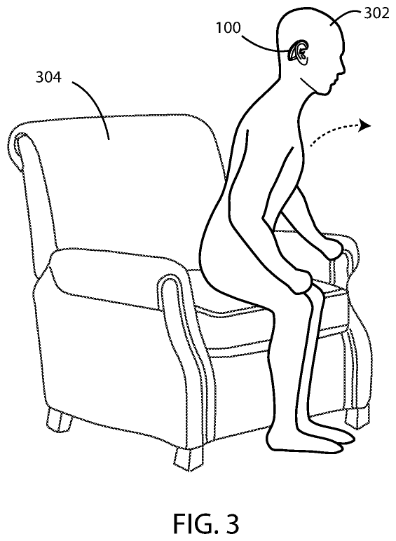

Referring now to FIG. 3, a schematic view of a device wearer 302 with an ear-wearable device 100 and rising from a chair 304 is shown in accordance with various embodiments herein. It will be appreciated that there are various ways of detecting a postural transition to a standing posture or position. Typically, assuming a standing posture will produce characteristic motion sensor signals associated with the vertical motion that occurs while transitioning to a standing posture. In some cases, the device can detect a transition to a standing posture by evaluating sensor signals to identify vertical motion.

However, detection of vertical motion by itself may lead to false positive scenarios. As such, in various embodiments, devices herein can look for a specific sequence of characteristic motions or the lack thereof to identify a transition to a standing posture. For example, transitioning between one posture and another will frequently include a characteristic tipping of the head forward which produces a detectable and characteristic signal as captured by a motion sensor herein. Therefore, in some embodiments, the device can evaluate sensor signals to identify head tipping followed by vertical motion to identify a transition to a standing posture. As another example, lying down or sitting down generally involves a lack of horizontal spatial motion in contrast to another activity such as walking. Thus, in some embodiments, the device can evaluate sensor signals to identify a lack of substantial horizontal spatial motion followed by vertical motion to identify a transition to a standing posture. In some embodiments, multiple characteristics can be combined to identify a transition to a standing posture. For example, in some embodiments the device can be configured to evaluate sensor signals to identify a lack of substantial horizontal spatial motion, followed by head tipping, followed by vertical motion to identify a transition to a standing posture.

Further, in various embodiments, a transition to a standing posture can be identified by matching signals from one or more sensors against one or more stored templates or patterns. For example, one or more templates or patterns reflecting a transition to a standing posture (a positive example) can stored by the system and/or one or more templates or patterns reflecting the absence of a transition to a standing posture (a negative example) can be stored by the system and then current sensor signals can be matched against the template(s) or pattern(s) to determine whether a transition to standing position or posture is likely occurring or not. In some embodiments, as part of a set-up or calibration operation, the device can prompt the device wearer to assume a standing position and record signals from the sensors during the transition to standing. The recorded signals reflecting a transition to a standing position or posture for that individual device wearer can then be used as a template or exemplary pattern to match future sensor signals against in order to positively identify a transition to a standing position or posture. Details of pattern matching techniques are described in greater detail below.

A starting posture of the device wearer before they transition to a standing position or posture may impact the values measured by sensors of devices herein. For example, for some individuals a transition from lying down to standing may be more likely to cause detectable features of orthostatic intolerance conditions than other postural transitions. Thus, it can be significant to determine the starting position or posture of the device wearer. As such, in various embodiments, the ear-wearable device 100 can be configured to process signals from the motion sensor to determine a starting posture prior to transition of the device wearer to a standing position. For example, the device can evaluate signals of a motion sensor over a lookback period when a transition to a standing position is detected to characterize the starting position or posture. As a specific example, the direction of gravity can be determined by evaluating the signals of an accelerometer that can be part of various embodiments herein. Further, the position of the head can be indicative as to whether the individual is lying down or sitting down. Because the devices herein are ear-wearable, signals from an accelerometer thereof can be used to determine head position and distinguish between a device wearer lying down or sitting down.

In some embodiments, devices herein can be configured to interface with and/or receive signals from other devices that can mark a postural transition to postural transition to a standing posture or position and/or the beginning of such a postural transition. For example, a chair or a bed can be equipped with a load cell or another sensor that can produce a characteristic signal when a device wearer rises from the same. The chair or bed can pass a signal onto the ear-wearable device or another component of the system indicating that the device wearer has transitioned to a standing posture and/or begun the process of transitioning. Devices and systems herein can also interface with other devices that can detect and/or assist in detecting that the device wearer has transitioned to a standing posture and/or begun the process of transitioning.

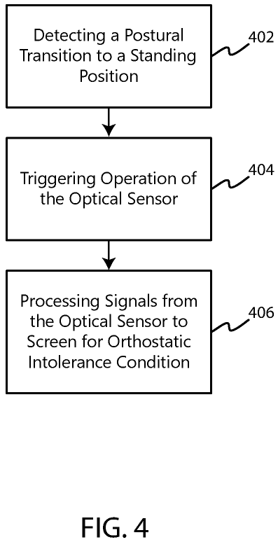

Referring now to FIG. 4, a flowchart of operations is shown in accordance with various embodiments herein. A method for screening for orthostatic intolerance conditions using an ear-wearable system can include an operation a processing signals from a motion sensor to detect a postural transition of a device wearer to a standing position 402, details of which have been described with respect to FIG. 3 and elsewhere herein. The method for screening for orthostatic intolerance conditions using an ear-wearable system can also include an operation of triggering an optical sensor 404 after a transition to a standing position has been detected. In various embodiments, triggering the optical sensor can include turning on a light emitter that is a part of an optical sensor herein.

The method for screening for orthostatic intolerance conditions using an ear-wearable system can also include an operation of processing signals from the optical sensor to screen for an orthostatic intolerance condition 406. It will be appreciated that orthostatic intolerance conditions can impact various measurable properties of the device wearer. In specific, orthostatic intolerance conditions can impact various measurable properties of the device wearer as detectable with an optical sensor. Thus, in various embodiments, the ear-wearable device 100 can be configured to process signals from an optical sensor and/or other sensors to screen for an orthostatic intolerance condition. For example, in various embodiments, the ear-wearable device 100 can be configured to process signals from the optical sensor and/or other sensors to detect one or more of blood pressure changes, blood flow morphology changes, inter-beat interval changes, pulse rate changes, and the like.

Specifically, in various embodiments, the ear-wearable device 100 can be configured to evaluate aspects of inter-beat (heart) interval (IBI) using various statistical analysis techniques. IBI can be derived by evaluating the morphology of a signal, such as that from an optical sensor herein or an electrical sensor, and measuring the time interval between repeating signal features, such as characteristic peaks corresponding to specific stages of the cardiac cycle. In various embodiments, IBI can be measured using an optical sensor signal (such as a PPG signal) peak-to-peak at a peak of blood volume or blood flow. In some embodiments, the ear-wearable device 100 can be configured to evaluate variability in inter-beat (heart) interval (IBI) changes. In some embodiments, the ear-wearable device 100 can be configured to evaluate the difference or delta between pairs or triplets in observed peaks and run statistical functions on the deltas. Many different standard statistical analysis techniques can be applied herein.

In various embodiments, the ear-wearable device 100 can be configured to identify changes in inter-beat interval changes that can be indicative of an orthostatic intolerance condition. In various embodiments, the ear-wearable device 100 can be configured to identify decreased variability in inter-beat interval changes as indicative of an orthostatic intolerance condition.

In various embodiments, the ear-wearable device 100 can be configured to process signals from the optical sensor to determine a blood flow morphology and/or changes in the same. Blood flow morphology as derived from a sensor signal herein can include characteristic peaks and valleys, magnitude of the same, width of the same, slopes of the same portions interconnecting characteristic peaks and valleys, and the like. In various embodiments, the ear-wearable device 100 can be configured to process signals from the optical sensor to determine a blood flow morphology consistent with reduced systolic blood pressure.

In various embodiments, the ear-wearable device 100 can be configured to process signals from the optical sensor, and electrical sensor, or another sensor to detect pulse rate changes. This can be done by evaluating the morphology of a signal, such as that from an optical sensor herein or an electrical sensor, and measuring the number of repeating signal features, such as characteristic peaks corresponding to specific stages of the cardiac cycle over a given time period then converting the same into beats per unit time.

Pulse rate can be particularly significant as tachycardia (high pulse rate) is part of POTS (postural orthostatic tachycardia syndrome). However, a pulse rate that is only moderately above normal may not be indicative of POTS. As such, in various embodiments, the ear-wearable device 100 can be configured to process signals from the optical sensor to detect pulse rate changes exceeding a threshold value. The threshold value can be set based on standard guidelines for all individuals, standard guidelines as classified by age, gender, etc., to a custom value by a medical professional, automatically set based on data gathered previously for the individual device wearer, or in another way. In some embodiments, a threshold value of pulse rate increase for POTS can be a specific increase over a preexisting heart rate before transitioning to a standing posture. For example, a threshold value of pulse rate increase for POTS can be an increase of 20, 25, 30, 35, 40 or more beats per minute, or an amount falling within a range between any of the foregoing.

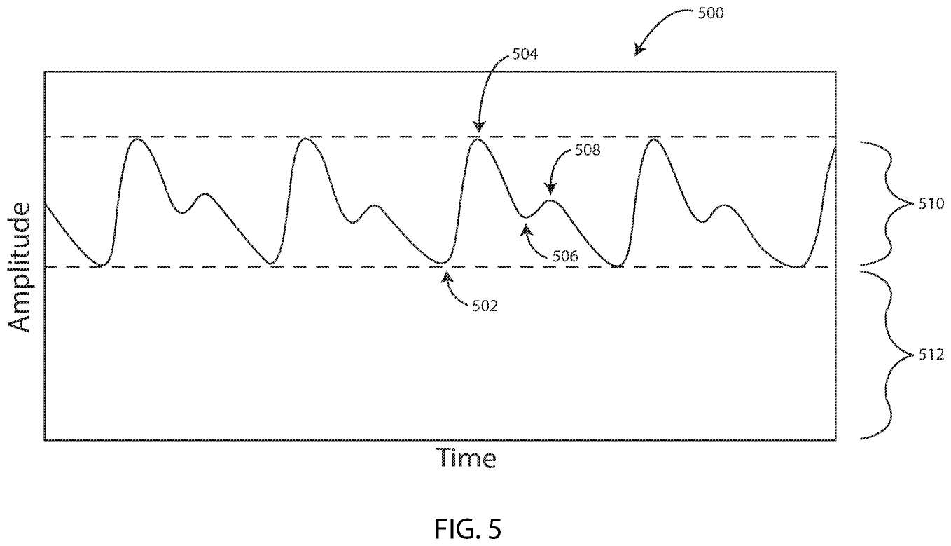

Referring now to FIG. 5, an exemplary view of an optical sensor signal 500 is shown in accordance with various embodiments herein. The optical sensor signal 500 includes a pulse onset 502. The optical sensor signal 500 also includes a systolic peak 504. The optical sensor signal 500 also includes a dicrotic notch 506. The optical sensor signal 500 also includes a diastolic peak 508.

The optical sensor signal 500 can includes an AC portion 510 as well as a DC portion 512. The DC portion 512 of the signal is generally attributable to the bulk absorption of the skin tissue, while the AC portion 510 is attributable to variation in blood volume in the skin caused by the pressure pulse of the cardiac cycle. The height of AC portion 510 of the signal is proportional to the pulse pressure, the difference between the systolic and diastolic pressure in the arteries. Changes in the height or magnitude of the AC portion 512 of the signal over time can be correlated with changes in blood pressure. Thus, changes in the AC portion 512 of the optical sensor signal 500 after a transition to standing posture can be used to screen for orthostatic intolerance conditions herein.

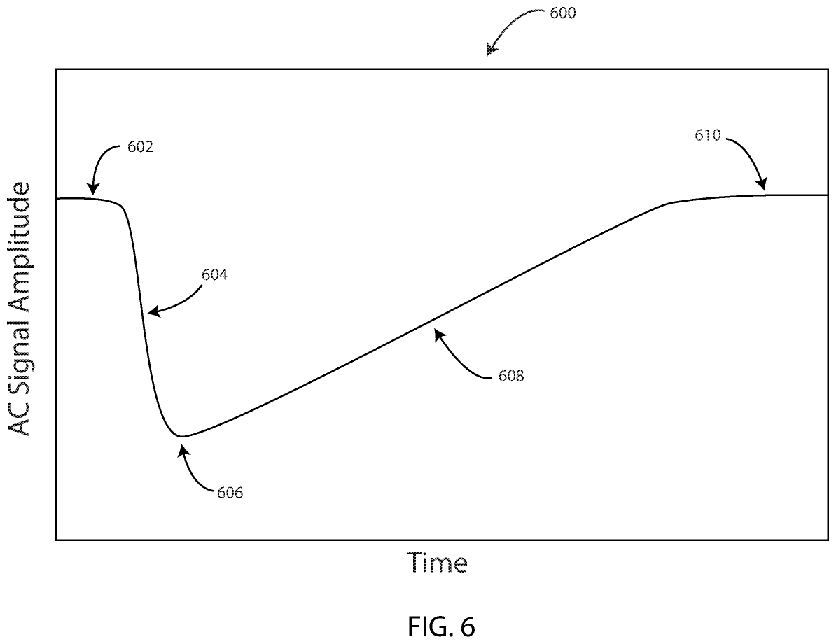

Referring now to FIG. 6, an exemplary view of the amplitude of the AC portion of an optical sensor signal over time consistent with an orthostatic intolerance condition is shown in accordance with various embodiments herein. The signal 600 includes a starting value 602 that can be correlated to a starting blood pressure. The signal 600 also includes a drop 604. The drop 604 can occur during or shortly after a transition to standing position or posture. The signal 600 also includes a level of maximum depression 606. The magnitude of the maximum depression 606 can be indicative of the severity of the orthostatic intolerance condition for the individual. Changes in the maximum depression 606 over time may reflect a change in health status of the individual. As such, tracking of maximum depression 606 over time can be useful for medical professionals and can thus be tracked and stored by devices herein.

The signal 600 also includes a recovery 608. The signal 600 also includes a recovered value 610. In some embodiments, the speed, and/or duration, and/or slope of the recovery 608 may reflect a change in health status of the individual. As such, tracking of recovery 608 over time can be useful for medical professionals and can thus be tracked and stored by devices herein.

In various embodiments, the ear-wearable device 100 can be configured to process signals from the optical sensor to determine a magnitude of change in the AC portion of the optical sensor signal over time, which can be correlated with a change in blood pressure over time, such as a change in systolic blood pressure. In various embodiments, the ear-wearable device 100 can be configured to process signals from the optical sensor to determine a recovery rate in the magnitude of the AC portion of the signal and/or blood pressure to normal levels. In various embodiments, the ear-wearable device 100 can be configured to process signals from the optical sensor to determine a magnitude and duration of change in AC portion of the signal and/or blood pressure.

In various embodiments, the device wearer 302 can be monitored by the device or system after they have assumed a standing position and, in some cases, even after their blood pressure has returned to a normal level. This can provide insight into their health status and be useful for assessing the likelihood of them experiencing a fall or other accident. For example, the device or system can characterize a degree of postural sway exhibited by the device wearer.

In some embodiments, the ear-wearable device 100 can be configured to prompt the device wearer 302 to take a precautionary action if an orthostatic intolerance condition is detected and/or a condition such as significant postural sway is detected. In various embodiments, the precautionary action can include instructing the device wearer to sit back down. In various embodiments, the precautionary action can include instructing the device wearer to use an assistive device, such as a cane, walking stabilizer, hand railing, or the like. In various embodiments, the precautionary action can include performing an exercise.

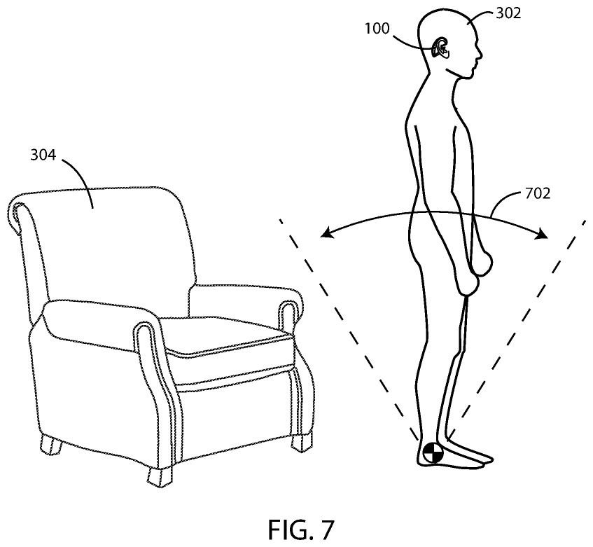

Referring now to FIG. 7, a schematic view of a device wearer 302 with an ear-wearable device 100 in a standing posture after having risen from a chair 304 is shown in accordance with various embodiments herein. The device wearer 302 is shown exhibiting a degree of postural sway 702. In various embodiments, the ear-wearable device 100 can be configured to process signals from the motion sensor to detect postural sway 702 after the device wearer 302 assumes a standing position. In various embodiments, the ear-wearable device 100 can be configured to issue a warning to the device wearer 302 if postural sway 702 crossing a threshold value can be detected.

In various embodiments, the ear-wearable device 100 can be configured to evaluate the device wearer and/or query a device wearer regarding their condition after they assume a standing position. For example, the ear-wearable device 100 can be configured to query a device wearer (either directly or indirectly by using an accessory device) regarding their condition after they assume a standing position. As another example, the ear-wearable device 100 can be configured to evaluate the device wearer by evaluating a reaction speed of a device wearer after they assume a standing position. For example, the device or system can prompt the device wearer to take an action and then measure the amount of time it takes them to begin the same. If their reaction speed is slowed compared with a normal or baseline value, this may be an additional indication that they suffer from an orthostatic intolerance condition.

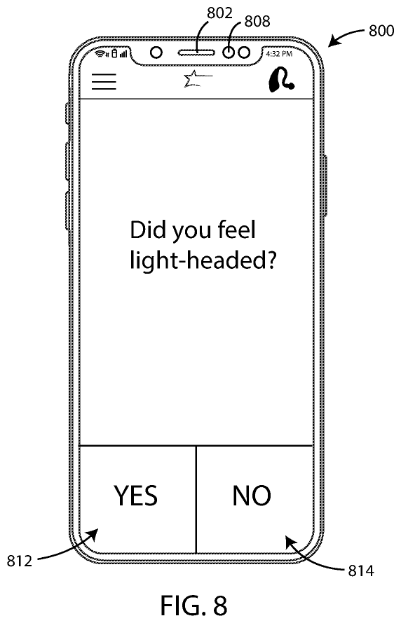

Accessory devices can be used in accordance with device and systems herein and can include various components. Referring now to FIG. 8, a schematic view of an accessory device 800 is shown in accordance with various embodiments herein. The accessory device 800 includes a speaker 802. The accessory device 800 also includes a camera 808. The accessory device 800 also includes a user input object 812. The accessory device 800 also includes a second user input object 814. In this example, the ear-wearable device can be configured to utilize the accessory device 800 to query the device wearer 302 regarding their condition after they assume a standing position. The accessory device 800 can receive feedback from the device wearer 302 and convey the same back to the ear-wearable device.

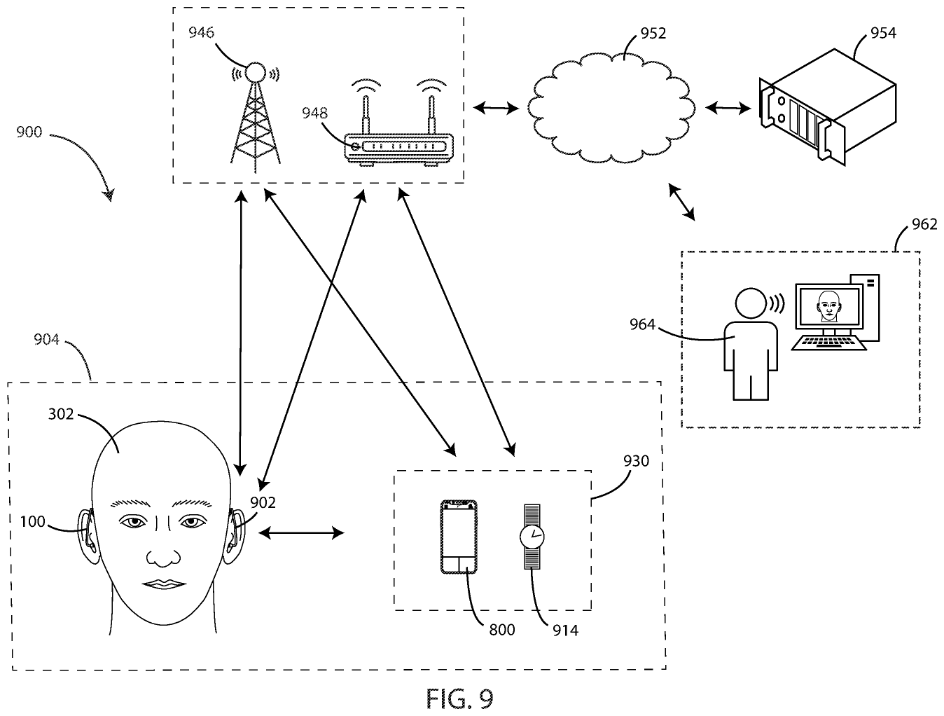

Referring now to FIG. 9, a schematic view of a system is shown in accordance with various embodiments herein. FIG. 9 shows a device wearer 302 with an ear-wearable device 100 and a second ear-wearable device 902. The device wearer 302 is at a first location or patient location 904. The system can include and/or can interface with other devices 930 at the first location 904. The other devices 930 in this example can include an accessory device 800, which could be a smart phone or similar mobile communication/computing device in some embodiments. The other devices 930 in this example can also include a wearable device 914, which could be an external wearable device 914 such as a smart watch or the like.

FIG. 9 also shows communication equipment including a cell tower 946 and a network router 948. FIG. 9 also schematically depicts the cloud 952 or similar data communication network. FIG. 9 also depicts a cloud computing resource 954. The communication equipment can provide data communication capabilities between the ear-wearable devices 100, 902 and other components of the system and/or components such as the cloud 952 and cloud resources such as a cloud computing resource 954. In some embodiments, the cloud 952 and/or resources thereof can host an electronic medical records system. In some embodiments, the cloud 952 can provide a link to an electronic medical records system.

FIG. 9 also shows a remote location 962. The remote location 962 can be the site of a third party 964, which can be a medical professional, care provider, loved one, or the like. The third party 964 can receive reports regarding the identified orthostatic intolerance conditions of the device wearer and/or details regarding the same. Specifically, in various embodiments, the ear-wearable device 100 can be configured to send an alert to a care provider if a possible orthostatic intolerance condition is detected. In some embodiments, the third party 964 can provide instructions for the device wearer regarding actions to take, such as actions to reduce or alleviate an orthostatic intolerance condition, instructions to enhance their safety in view of experiencing an orthostatic intolerance condition, instructions with respect to medications that may impact an orthostatic intolerance condition, and the like.

In various embodiments, the ear-wearable stress monitoring system 900 can be configured to send information regarding an orthostatic intolerance condition to an electronic medical record system. In various embodiments, the ear-wearable stress monitoring system 900 can be configured to send information regarding an orthostatic intolerance condition to a third party 964. In some embodiments, the ear-wearable stress monitoring system 900 can be configured to receive information regarding an orthostatic intolerance condition as relevant to the individual through an electronic medical record system. Such received information can be used alongside data from microphones and other sensors herein and/or incorporated into machine learning classification models used herein.

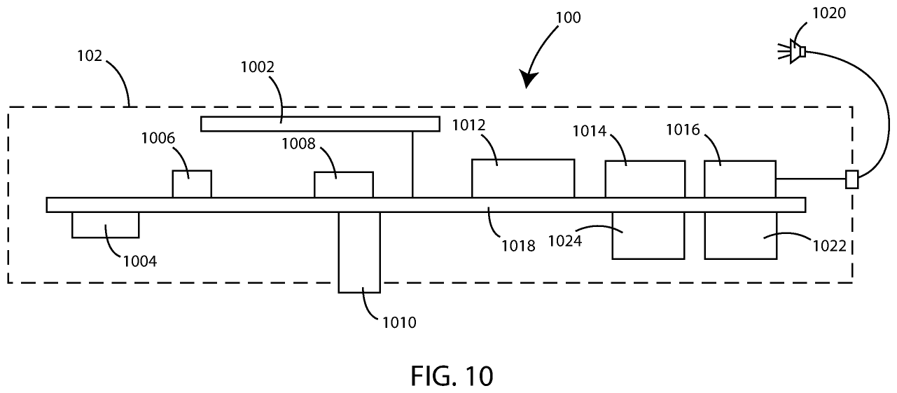

Referring now to FIG. 10, a schematic block diagram is shown with various components of an ear-wearable device 100 in accordance with various embodiments. The block diagram of FIG. 10 represents a generic ear-wearable device for purposes of illustration. The ear-wearable device 100 shown in FIG. 10 includes several components electrically connected to a flexible mother circuit 1018 (e.g., flexible mother board) which is disposed within housing 102. A power supply circuit 1004 can include a battery and can be electrically connected to the flexible mother circuit 1018 and provides power to the various components of the ear-wearable device 100. One or more microphones 1006 are electrically connected to the flexible mother circuit 1018, which provides electrical communication between the microphones 1006 and a digital signal processor (DSP) 1012. Microphones herein can be of various types including, but not limited to, unidirectional, omnidirectional, MEMS based microphones, piezoelectric microphones, magnetic microphones, electret condenser microphones, and the like. Among other components, the DSP 1012 incorporates or is coupled to audio signal processing circuitry configured to implement various functions described herein. A sensor package 1014 can be coupled to the DSP 1012 via the flexible mother circuit 1018. The sensor package 1014 can include one or more different specific types of sensors such as those described in greater detail below. One or more user switches 1010 (e.g., on/off, volume, mic directional settings) are electrically coupled to the DSP 1012 via the flexible mother circuit 1018. It will be appreciated that the user switches 1010 can extend outside of the housing 102.

An audio output device 1016 is electrically connected to the DSP 1012 via the flexible mother circuit 1018. In some embodiments, the audio output device 1016 comprises a speaker (coupled to an amplifier). In other embodiments, the audio output device 1016 comprises an amplifier coupled to an external receiver 1020 adapted for positioning within an ear of a wearer. The external receiver 1020 can include an electroacoustic transducer, speaker, or loudspeaker. The ear-wearable device 100 may incorporate a communication device 1008 coupled to the flexible mother circuit 1018 and to an antenna 1002 directly or indirectly via the flexible mother circuit 1018. The communication device 1008 can be a BLUETOOTH® transceiver, such as a BLE (BLUETOOTH® low energy) transceiver or other transceiver(s) (e.g., an IEEE 802.11 compliant device). The communication device 1008 can be configured to communicate with one or more external devices, such as those discussed previously, in accordance with various embodiments. In various embodiments, the communication device 1008 can be configured to communicate with an external visual display device such as a smart phone, a video display screen, a tablet, a computer, or the like.

In various embodiments, the ear-wearable device 100 can also include a control circuit 1022 and a memory storage device 1024. The control circuit 1022 can be in electrical communication with other components of the device. In some embodiments, a clock circuit can be in electrical communication with the control circuit. The control circuit 1022 can execute various operations, such as those described herein. In various embodiments, the control circuit 1022 can execute operations resulting in the provision of a user input interface by which the ear-wearable device 100 can receive inputs (including audible inputs, touch based inputs, and the like) from the device wearer. The control circuit 1022 can include various components including, but not limited to, a microprocessor, a microcontroller, an FPGA (field-programmable gate array) processing device, an ASIC (application specific integrated circuit), or the like. The memory storage device 1024 can include both volatile and non-volatile memory. The memory storage device 1024 can include ROM, RAM, flash memory, EEPROM, SSD devices, NAND chips, and the like. The memory storage device 1024 can be used to store data from sensors as described herein and/or processed data generated using data from sensors as described herein.

It will be appreciated that various of the components described in FIG. 10 can be associated with separate devices and/or accessory devices to the ear-wearable device. By way of example, microphones can be associated with separate devices and/or accessory devices. Similarly, audio output devices can be associated with separate devices and/or accessory devices to the ear-wearable device. Further accessory devices as discussed herein can include various of the components as described with respect to an ear-wearable device. For example, an accessory device can include a control circuit, a microphone, a motion sensor, and a power supply, amongst other things.

Accessory devices herein can include various different components. In some embodiments, the accessory device can be a personal communications device, such as a smart phone. However, the accessory device can also be other things such as a secondary wearable device, a handheld computing device, a dedicated location determining device (such as a handheld GPS unit), or the like.

### Methods

Many different methods are contemplated herein, including, but not limited to, methods of making, methods of using, and the like. Aspects of system/device operation described elsewhere herein can be performed as operations of one or more methods in accordance with various embodiments herein.

In an embodiment, a method for screening for orthostatic intolerance conditions using an ear-wearable system is included, the method processing signals from a motion sensor to detect a postural transition of a device wearer to a standing position, triggering operation of the optical sensor, and processing signals from the optical sensor to screen for an orthostatic intolerance condition. In various embodiments, the orthostatic intolerance condition can include a condition selected from the group consisting of postural orthostatic tachycardia syndrome and orthostatic hypotension.

In an embodiment, the method can further include processing signals from the optical sensor to detect inter-beat interval changes. In an embodiment, the method can further include processing signals from the optical sensor to detect pulse rate changes. In an embodiment, the method can further include processing signals from the optical sensor to detect pulse rate changes exceeding a threshold value. In an embodiment, the method can further include processing signals from the optical sensor to determine a blood flow morphology. In an embodiment, the method can further include processing signals from the optical sensor to determine a blood flow morphology consistent with reduced systolic blood pressure and/or an orthostatic intolerance condition event. In an embodiment, the method can further include processing signals from the optical sensor to determine a magnitude of change in the AC portion of the signal which can be correlated with a magnitude of change in systolic blood pressure. In an embodiment, the method can further include processing signals from the optical sensor to determine a recovery rate in the AC portion of the signal which can be correlated with a recovery rate of systolic blood pressure to normal levels. In an embodiment, the method can further include processing signals from the optical sensor to determine a magnitude and duration of change in the AC portion of the signal which can be correlated with a magnitude and duration of change in systolic blood pressure.

In an embodiment, the method can further include issuing a command to an accessory device to prompt the device wearer to stand. In an embodiment, the method can further include querying the device wearer regarding their condition after they assume a standing position. In an embodiment, the method can further include evaluating a reaction speed of the device wearer after they assume a standing position.

In an embodiment, the method can further include sending an alert to a care provider if a possible orthostatic intolerance condition is detected.

In an embodiment, the method can further include detecting postural sway after the device wearer assumes a standing position.

In an embodiment, the method can further include processing signals from the motion sensor to determine a starting posture prior to transition of the device wearer to a standing position.

In an embodiment, the method can further include triggering operation of the optical sensor after detection of the postural transition of the device wearer to the standing position when the postural transition was preceded by lying or sitting for at least a threshold amount of time.

In an embodiment, the method can further include warning the device wearer if an orthostatic intolerance condition is detected. In an embodiment, the method can further include warning the device wearer after detecting a sedentary period of the device wearer if orthostatic intolerance conditions were previously detected. In an embodiment, the method can further include warning the device wearer after detecting the beginning of a transition to a standing posture if orthostatic intolerance conditions were previously detected.

In an embodiment, the method can further include adjusting a fall risk threshold if an orthostatic intolerance condition is detected.

In an embodiment, the method can further include prompting the device wearer to take a precautionary action if an orthostatic intolerance condition is detected. In an embodiment, the method can further include prompting the device wearer to drink water if an orthostatic intolerance condition is detected.

In an embodiment, the method can further include providing stimulation to the device wearer when a postural transition to a standing position is detected to mitigate the effects of an orthostatic intolerance condition.

In an embodiment, the method can further include evaluating a signal from a temperature sensor while screening for an orthostatic intolerance condition. In an embodiment, the method can further include evaluating a signal from a temperature sensor and suspend screening for the orthostatic intolerance condition if the temperature sensor indicates a temperature falling outside of a predetermined range.

In an embodiment, the method can further include processing signals from the motion sensor over a time period prior to transition of the device wearer to a standing position to characterize an activity level of the device wearer.

In an embodiment, the method can further include setting a threshold value for detection of an orthostatic intolerance condition based at least in part on at least one factor of the device wearer selected from age, known diagnosis, BMI, footstep speed, known gait instability, known history of orthostatic intolerance conditions, and trends thereof. In an embodiment, the method can further include setting a threshold value for detection of an orthostatic intolerance condition based at least in part on known alcohol use, cardiovascular disease status, allergies, blood disorders, and prescribed medications.

### Pattern Identification and Matching

It will be appreciated that in various embodiments herein, a device or a system can be used to detect a pattern or patterns indicative of a postural transition to a standing position or posture, a change in condition and/or sensor signals reflecting an orthostatic intolerance condition, or the like. Such patterns can be detected in various ways. Some techniques are described elsewhere herein, but some further examples will now be described.

As merely one example, one or more sensors can be operatively connected to a controller (such as the control circuit described in FIG. 10) or another processing resource (such as a processor of another device or a processing resource in the cloud). The controller or other processing resource can be adapted to receive data from one or more of the sensors and/or process the same to identify properties or conditions of the subject over a monitoring time period based upon the data received from the sensor(s). As used herein, the term “data” can include a single datum or a plurality of data values or statistics. The term “statistics” can include any appropriate mathematical calculation or metric relative to data interpretation, e.g., probability, confidence interval, distribution, range, or the like. Further, as used herein, the term “monitoring time period” means a period of time over which characteristics of the subject are measured and statistics are determined. The monitoring time period can be any suitable length of time, e.g., 1 millisecond, 1 second, 10 seconds, 30 seconds, 1 minute, 10 minutes, 30 minutes, 1 hour, etc., or a range of time between any of the foregoing time periods.

Any suitable technique or techniques can be utilized to determine statistics for the various data from the sensors, e.g., direct statistical analyses of time series data from the sensors, differential statistics, comparisons to baseline or statistical models of similar data, etc. Such techniques can be general or individual-specific and represent long-term or short-term behavior. These techniques could include standard pattern classification methods such as Gaussian mixture models, clustering as well as Bayesian approaches, machine learning approaches such as neural network models and deep learning, and the like.

Further, in some embodiments, the controller can be adapted to compare data, data features, and/or statistics against various other patterns, which could be prerecorded postural transition movement patterns or sensor signal patterns (baseline patterns) of the particular individual wearing an ear-wearable device herein, prerecorded postural transition movement patterns or sensor signal patterns (group baseline patterns) of a group of individuals wearing ear-wearable devices herein, one or more predetermined postural transition movement patterns or sensor signal patterns that serve as patterns indicative of an occurrence of a particular posture or postural transition or orthostatic intolerance condition (positive example patterns), one or more predetermined postural transition movement patterns or sensor signal patterns that serve as patterns indicative of the absence of a particular posture or postural transition or orthostatic intolerance condition (negative example patterns), or the like. As merely one scenario, if a postural transition movement patterns or sensor signal pattern is detected in an individual that exhibits similarity crossing a threshold value to a particular positive example pattern or substantial similarity to that pattern, wherein the pattern is specific for a particular postural transition or orthostatic intolerance condition state, then that can be taken as an indication of an occurrence of a particular postural transition or orthostatic intolerance condition state.

Similarity and dissimilarity can be measured directly via standard statistical metrics such normalized Z-score, or similar multidimensional distance measures (e.g., Mahalanobis or Bhattacharyya distance metrics), or through similarities of modeled data and machine learning. These techniques can include standard pattern classification methods such as Gaussian mixture models, clustering as well as Bayesian approaches, neural network models, and deep learning.

As used herein the term “substantially similar” means that, upon comparison, the sensor data are congruent or have statistics fitting the same statistical model, each with an acceptable degree of confidence. The threshold for the acceptability of a confidence statistic may vary depending upon the subject, sensor, sensor arrangement, type of data, context, condition, etc.

The statistics associated with the postural transition or orthostatic intolerance condition state of an individual over the monitoring time period can be determined by utilizing any suitable technique or techniques, e.g., standard pattern classification methods such as Gaussian mixture models, clustering, hidden Markov models, as well as Bayesian approaches, neural network models, and deep learning.

Various embodiments herein specifically include the application of a machine learning classification model. In various embodiments, the ear-wearable devices and/or systems herein can be configured to periodically update the machine learning classification model based on the postures, postural transitions or orthostatic intolerance condition states of the device wearer.

In some embodiments, a training set of data can be used in order to generate a machine learning classification model. The input data can include microphone and/or sensor data as described herein as tagged/labeled with binary and/or non-binary classifications of postures, postural transitions or orthostatic intolerance condition states. Binary classification approaches can utilize techniques including, but not limited to, logistic regression, k-nearest neighbors, decision trees, support vector machine approaches, naive Bayes techniques, and the like. Multi-class classification approaches (e.g., for non-binary classifications of postures, postural transitions or orthostatic intolerance condition states) can include k-nearest neighbors, decision trees, naive Bayes approaches, random forest approaches, and gradient boosting approaches amongst others.

In some embodiments, to facilitate a supervised machine learning approach, a device wearer can be put through a particular movement protocol (such as a particular postural movement protocol) in order to provide a training set of data that is specific for the device wearer. In some embodiments, a training set of data specific for the device wearer can be gathered as part of a fitting procedure associated with the device wearer getting the device(s). However, in other embodiments, unsupervised machine learning approaches can also be used.

In various embodiments, the device and/or system herein is configured to execute operations to generate or update the machine learning model on the ear-wearable device itself. In some embodiments, the ear-wearable device may convey data to another device such as an accessory device or a cloud computing resource in order to execute operations to generate or update a machine learning model herein. In various embodiments herein, threshold values used herein (as described at various points herein) can be calculated or otherwise derived through analysis of data regarding the device wearer. For example, in some embodiments, a threshold value can be set through evaluation of previous events related to the postures, postural transitions or orthostatic intolerance condition states of the device wearer. In some cases, such events can be detected by the ear-wearable device(s). In other cases, such events can be provided as input to the ear-wearable device(s) from another system, device, or third party. In some embodiments, the threshold value can be related to the occurrence of such events. In some embodiments, the threshold value can be related to the prediction of the occurrence of such events based on a comparison of past sensor data associated with the occurrence of such events and current sensor data. In some embodiments, the threshold value can divide categories of relevance for postures, postural transitions or orthostatic intolerance condition states such that a process of categorization also calculates threshold value(s). Categorization and/or calculation of threshold values can, in some cases, be performed using a machine learning approach including for example, an unsupervised machine learning approach. However, in some scenarios, supervised machine learning approaches can also be used. In some embodiments, calculation of threshold values can be performed using statistical approaches.

It will be appreciated that processing herein can be performed by various devices. For example, processing associated with pattern identification and matching can be performed by the ear-wearable device(s), by one or more accessory devices, or in the cloud. In some cases, processing associated with pattern identification and matching can be performed by multiple devices or layers of the system.

### Sensor Package

Various embodiments herein include one or more sensors. Specifically, devices and systems herein can include one or more sensors (including one or more discrete or integrated sensors) to provide data for use with operations to evaluate and/or characterize the status of a device wearer, detect a transition to a standing position or posture, and/or an effect of assuming a standing position or posture consistent with an orthostatic intolerance condition. Further details about the sensors are provided as follows. However, it will be appreciated that this is merely provided by way of example and that further variations are contemplated herein. Also, it will be appreciated that a single sensor may provide more than one type of physiological data. For example, heart rate, respiration, blood pressure, or any combination thereof may be extracted from PPG (photoplethysmography) optical sensor data.

In various embodiments, a transition to a standing position or posture by the device wearer is detected using data produced by at least one of the motion sensor and the microphone. In various embodiments, an effect of assuming a standing position or posture consistent with an orthostatic intolerance condition is detected using data produced by at least an optical sensor, such as an optical PPG (photoplethysmography) sensor. In various embodiments, other sensors can also be included such as at least one of a heart rate sensor, a heart rate variability sensor, an electrocardiogram (ECG) sensor, a blood oxygen sensor, a blood pressure sensor, a skin conductance sensor, a temperature sensor (such as a core body temperature sensor, skin temperature sensor, ear-canal temperature sensor, or another temperature sensor), an electroencephalograph (EEG) sensor, and a respiratory sensor.

Motion sensors herein can include inertial measurement units (IMU), accelerometers, gyroscopes, barometers, altimeters, and the like. The IMU can be of a type disclosed in commonly owned U.S. patent application Ser. No. 15/331,230, filed Oct. 21, 2016, which is incorporated herein by reference. As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational rate. In some embodiments, an IMU can also include a magnetometer to detect a magnetic field.

Optical PPG (photoplethysmography) sensors herein can include one or more light emitters or light sources (such as light emitting diodes or other light emitting components) and one or more light detectors or photodetectors (such as a phototransistor, photodiode, photoresistor, or other light detecting components). In some embodiments, the light emitter can emit light at a near infrared frequency or frequencies.

In some embodiments, electromagnetic communication radios or electromagnetic field sensors (e.g., telecoil, NFMI, TMR, GMR, etc.) sensors may be used to detect motion or changes in position. In some embodiments, biometric sensors may be used to detect body motions or physical activity. Motion sensors can be used to track movements of a patient in accordance with various embodiments herein.

In some embodiments, the motion sensors can be disposed in a fixed position with respect to the head of a patient, such as worn on or near the head or ears. In some embodiments, operatively connected motion sensors can be worn on or near another part of the body such as on a wrist, arm, or leg of the patient.

According to various embodiments, sensors herein can include one or more of an IMU, and accelerometer (3, 6, or 9 axis), a gyroscope, a barometer, an altimeter, a magnetometer, a magnetic sensor, an eye movement sensor, a pressure sensor, an acoustic sensor, a telecoil, a heart rate sensor, a global positioning system (GPS) circuit, a temperature sensor, a blood pressure sensor, an oxygen saturation sensor, an optical sensor, a blood glucose sensor (optical or otherwise), a galvanic skin response sensor, a cortisol level sensor (optical or otherwise), a microphone, acoustic sensor, an electrocardiogram (ECG) sensor, electroencephalography (EEG) sensor which can be a neurological sensor, eye movement sensor (e.g., electrooculogram (EOG) sensor), myographic potential electrode sensor (or electromyography—EMG), a heart rate monitor, a pulse oximeter or oxygen saturation sensor (SpO2), a wireless radio antenna, blood perfusion sensor, hydrometer, sweat sensor, cerumen sensor, air quality sensor, pupillometry sensor, cortisol level sensor, hematocrit sensor, light sensor, image sensor, and the like.

In some embodiments, sensors herein can be part of an ear-wearable device. However, in some embodiments, the sensors utilized can include one or more additional sensors that are external to an ear-wearable device. For example, various of the sensors described above can be part of a wrist-worn or ankle-worn sensor package, or a sensor package supported by a chest strap. In some embodiments, sensors herein can be disposable sensors that are adhered to the device wearer (“adhesive sensors”) and that provide data to the ear-wearable device or another component of the system.

Data produced by the sensor(s) herein can be operated on by a processor of the device or system.

As used herein, the term “microphone” shall include reference to all types of devices used to capture sounds including various types of microphones (including, but not limited to, carbon microphones, fiber optic microphones, dynamic microphones, electret microphones, ribbon microphones, laser microphones, condenser microphones, cardioid microphones, crystal microphones) and vibration sensors (including, but not limited to accelerometers and various types of pressure sensors). Microphones herein can include analog and digital microphones.

Systems herein can also include various signal processing chips and components such as analog-to-digital converters and digital-to-analog converters. Systems herein can operate with audio data that is gathered, transmitted, and/or processed reflecting various sampling rates. By way of example, sampling rates used herein can include 8,000 Hz, 11,025 Hz, 16,000 Hz, 22,050 Hz, 32,000 Hz, 37,800 Hz, 44,056 Hz, 44,100 Hz, 47,250 Hz, 48,000 Hz, 50,000 Hz, 50,400 Hz, 64,000 Hz, 88,200 Hz, 96,000 Hz, 176,400 Hz, 192,000 Hz, or higher or lower, or within a range falling between any of the foregoing. Audio data herein can reflect various bit depths including, but not limited to 8, 16, and 24-bit depth. Microphones herein can include both directional and omnidirectional microphones.

An eye movement sensor herein may be, for example, an electrooculographic (EOG) sensor, such as an EOG sensor disclosed in commonly owned U.S. Pat. No. 9,167,356, which is incorporated herein by reference. A pressure sensor herein can be, for example, a MEMS-based pressure sensor, a piezo-resistive pressure sensor, a flexion sensor, a strain sensor, a diaphragm-type sensor and the like.

A temperature sensor herein can be, for example, a thermistor (thermally sensitive resistor), a resistance temperature detector, a thermocouple, a semiconductor-based sensor, an infrared sensor, or the like.

A blood pressure sensor herein can be, for example, a pressure sensor. The heart rate sensor can be, for example, an electrical signal sensor, an acoustic sensor, a pressure sensor, an infrared sensor, an optical sensor, or the like.

An oxygen saturation sensor (such as a blood oximetry sensor) herein can be, for example, an optical sensor, an infrared sensor, a visible light sensor, or the like.

An electrical signal sensor herein can include two or more electrodes and can include circuitry to sense and record electrical signals including sensed electrical potentials and the magnitude thereof (according to Ohm's law where V=IR) as well as measure impedance from an applied electrical potential.

It will be appreciated that sensors herein can include one or more sensors that are external to the ear-wearable device. In addition to the external sensors discussed hereinabove, the sensor package can comprise a network of body sensors (such as those listed above) that sense movement of a multiplicity of body parts (e.g., arms, legs, torso). In some embodiments, the ear-wearable device can be in electronic communication with the sensors or processor of a medical device.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. An ear-wearable device comprising:
a control circuit;
a microphone, wherein the microphone is in electrical communication with the control circuit;
an electroacoustic transducer, wherein the electroacoustic transducer is in electrical communication with the control circuit; and
a sensor package, the sensor package comprising
a motion sensor; and
an optical sensor;
wherein the sensor package is in electrical communication with the control circuit;
wherein the ear-wearable device is configured to
process signals from the motion sensor to detect a postural transition of a device wearer to a standing position;
trigger operation of the optical sensor; and
process signals from the optical sensor to screen for an orthostatic intolerance condition.

2. The ear-wearable device of any of claims 1 and 3-41, the orthostatic intolerance condition comprising a condition selected from the group consisting of postural orthostatic tachycardia syndrome and orthostatic hypotension.

3. The ear-wearable device of any of claims 1-2 and 4-41, the motion sensor comprising at least one sensor selected from the group consisting of an accelerometer and a gyroscope.

4. The ear-wearable device of any of claims 1-3 and 5-41, wherein the ear-wearable device is configured to process signals from the optical sensor to detect inter-beat interval changes.

5. The ear-wearable device of any of claims 1-4 and 6-41, wherein the ear-wearable device is configured to identify decreased variability in inter-beat interval changes as indicative of an orthostatic intolerance condition.

6. The ear-wearable device of any of claims 1-5 and 7-41, wherein the ear-wearable device is configured to process signals from the optical sensor to detect pulse rate changes.

7. The ear-wearable device of any of claims 1-6 and 8-41, wherein the ear-wearable device is configured to process signals from the optical sensor to detect pulse rate changes exceeding a threshold value.

8. The ear-wearable device of any of claims 1-7 and 9-41, wherein the ear-wearable device is configured to process signals from the optical sensor to determine a blood flow morphology.

9. The ear-wearable device of any of claims 1-8 and 10-41, wherein the ear-wearable device is configured to process signals from the optical sensor to determine a blood flow morphology consistent with reduced systolic blood pressure.

10. The ear-wearable device of any of claims 1-9 and 11-41, wherein the ear-wearable device is configured to process signals from the optical sensor to determine a magnitude of change in an AC portion of the signals.

11. The ear-wearable device of any of claims 1-10 and 12-41, wherein the ear-wearable device is configured to process signals from the optical sensor to determine a recovery rate in the AC portion of the signals to normal levels.

12. The ear-wearable device of any of claims 1-11 and 13-41, wherein the ear-wearable device is configured to process signals from the optical sensor to determine a magnitude and duration of change in the AC portion of the signals.

13. The ear-wearable device of any of claims 1-12 and 14-41, the optical sensor comprising a light emitter.

14. The ear-wearable device of any of claims 1-13 and 15-41, wherein the light emitter emits light at a near-infrared wavelength.

15. The ear-wearable device of any of claims 1-14 and 16-41, wherein triggering operation of the optical sensor includes turning on the light emitter.

16. The ear-wearable device of any of claims 1-15 and 17-41, wherein the ear-wearable device is configured to prompt the device wearer to stand.

17. The ear-wearable device of any of claims 1-16 and 18-41, wherein the ear-wearable device is configured to issue a command to an accessory device to prompt the device wearer to stand.

18. The ear-wearable device of any of claims 1-17 and 19-41, wherein the ear-wearable device is configured to query the device wearer regarding their condition after they assume a standing position.

19. The ear-wearable device of any of claims 1-18 and 20-41, wherein the ear-wearable device is configured to evaluate a reaction speed of the device wearer after they assume a standing position.

20. The ear-wearable device of any of claims 1-19 and 21-41, wherein the ear-wearable device is configured to send an alert to a care provider if a possible orthostatic intolerance condition is detected.

21. The ear-wearable device of any of claims 1-20 and 22-41, wherein the ear-wearable device is configured to process signals from the motion sensor to detect postural sway after the device wearer assumes a standing position.

22. The ear-wearable device of any of claims 1-21 and 23-41, wherein the ear-wearable device is configured to issue a warning to the device wearer if postural sway crossing a threshold value is detected.

23. The ear-wearable device of any of claims 1-22 and 24-41, wherein the ear-wearable device is configured to process signals from the motion sensor to determine a starting posture prior to transition of the device wearer to a standing position.

24. The ear-wearable device of any of claims 1-23 and 25-41, wherein the ear-wearable device is configured to trigger operation of the optical sensor after detection of the postural transition of the device wearer to the standing position when the postural transition was preceded by lying or sitting for at least a threshold amount of time.

25. The ear-wearable device of any of claims 1-24 and 26-41, wherein the ear-wearable device is configured to warn the device wearer if an orthostatic intolerance condition is detected.

26. The ear-wearable device of any of claims 1-25 and 27-41, wherein the ear-wearable device is configured to warn the device wearer after detecting a sedentary period of the device wearer if orthostatic intolerance conditions were previously detected.

27. The ear-wearable device of any of claims 1-26 and 28-41, wherein the ear-wearable device is configured to warn the device wearer after detecting the beginning of a transition to a standing posture if orthostatic intolerance conditions were previously detected.

28. The ear-wearable device of any of claims 1-27 and 29-41, wherein the ear-wearable device is configured to adjust a fall risk threshold if an orthostatic intolerance condition is detected.

29. The ear-wearable device of any of claims 1-28 and 30-41, wherein the ear-wearable device is configured to prompt the device wearer to take a precautionary action if an orthostatic intolerance condition is detected.

30. The ear-wearable device of any of claims 1-29 and 31-41, the precautionary action comprising sitting back down.

31. The ear-wearable device of any of claims 1-30 and 32-41, the precautionary action comprising using an assistive device.

32. The ear-wearable device of any of claims 1-31 and 33-41, the precautionary action comprising performing an exercise.

33. The ear-wearable device of any of claims 1-32 and 34-41, wherein the ear-wearable device is configured to prompt the device wearer to drink water if an orthostatic intolerance condition is detected.

34. The ear-wearable device of any of claims 1-33 and 35-41, wherein the ear-wearable device is configured to provide stimulation to the device wearer when a postural transition to a standing position is detected to mitigate the effects of an orthostatic intolerance condition.

35. The ear-wearable device of any of claims 1-34 and 36-41, the sensor package comprising a temperature sensor.

36. The ear-wearable device of any of claims 1-35 and 37-41, wherein the ear-wearable device is configured to evaluate a signal from the temperature sensor while screening for an orthostatic intolerance condition.

37. The ear-wearable device of any of claims 1-36 and 38-41, wherein the ear-wearable device is configured to evaluate a signal from the temperature sensor and suspend screening for the orthostatic intolerance condition if the temperature sensor indicates a temperature falling outside of a predetermined range.

38. The ear-wearable device of any of claims 1-37 and 39-41, wherein the ear-wearable device is configured to process signals from the motion sensor over a time period prior to transition of the device wearer to a standing position to characterize an activity level of the device wearer.

39. The ear-wearable device of any of claims 1-38 and 40-41, wherein the ear-wearable device is configured to evaluate the activity level and suspend screening for the orthostatic intolerance condition if the activity level crosses a threshold value.

40. The ear-wearable device of any of claims 1-39 and 41, wherein the ear-wearable device is configured to set a threshold value for detection of an orthostatic intolerance condition based at least in part on at least one factor of the device wearer selected from age, known diagnosis, BMI, footstep speed, known gait instability, known history of orthostatic intolerance conditions, and trends thereof.

41. The ear-wearable device of any of claims 1-40, wherein the ear-wearable device is configured to set a threshold value for detection of an orthostatic intolerance condition based at least in part on known alcohol use, cardiovascular disease status, allergies, blood disorders, and prescribed medications.

42. A method for screening for orthostatic intolerance conditions using an ear-wearable system comprising:
processing signals from a motion sensor to detect a postural transition of a device wearer to a standing position;
triggering operation of the optical sensor; and
processing signals from the optical sensor to screen for an orthostatic intolerance condition.

43. The method of any of claims 42 and 44-70, the orthostatic intolerance condition comprising a condition selected from the group consisting of postural orthostatic tachycardia syndrome and orthostatic hypotension.

44. The method of any of claims 42-43 and 45-70, further comprising processing signals from the optical sensor to detect inter-beat interval changes.

45. The method of any of claims 42-44 and 46-70, further comprising processing signals from the optical sensor to detect pulse rate changes.

46. The method of any of claims 42-45 and 47-70, further comprising processing signals from the optical sensor to detect pulse rate changes exceeding a threshold value.

47. The method of any of claims 42-46 and 48-70, further comprising processing signals from the optical sensor to determine a blood flow morphology.

48. The method of any of claims 42-47 and 49-70, further comprising processing signals from the optical sensor to determine a blood flow morphology consistent with reduced systolic blood pressure.

49. The method of any of claims 42-48 and 50-70, further comprising processing signals from the optical sensor to determine a magnitude of change in the AC portion of the signals.

50. The method of any of claims 42-49 and 51-70, further comprising processing signals from the optical sensor to determine a recovery rate in the AC portion of the signals to normal levels.

51. The method of any of claims 42-50 and 52-70, further comprising processing signals from the optical sensor to determine a magnitude and duration of change in the AC portion of the signals.

52. The method of any of claims 42-51 and 53-70, further comprising issuing a command to an accessory device to prompt the device wearer to stand.

53. The method of any of claims 42-52 and 54-70, further comprising querying the device wearer regarding their condition after they assume a standing position.

54. The method of any of claims 42-53 and 55-70, further comprising evaluating a reaction speed of the device wearer after they assume a standing position.

55. The method of any of claims 42-54 and 56-70, further comprising sending an alert to a care provider if a possible orthostatic intolerance condition is detected.

56. The method of any of claims 42-55 and 57-70, further comprising detecting postural sway after the device wearer assumes a standing position.

57. The method of any of claims 42-56 and 58-70, further comprising processing signals from the motion sensor to determine a starting posture prior to transition of the device wearer to a standing position.

58. The method of any of claims 42-57 and 59-70, further comprising triggering operation of the optical sensor after detection of the postural transition of the device wearer to the standing position when the postural transition was preceded by lying or sitting for at least a threshold amount of time.

59. The method of any of claims 42-58 and 60-70, further comprising warning the device wearer if an orthostatic intolerance condition is detected.

60. The method of any of claims 42-59 and 61-70, further comprising warning the device wearer after detecting a sedentary period of the device wearer if orthostatic intolerance conditions were previously detected.

61. The method of any of claims 42-60 and 62-70, further comprising warning the device wearer after detecting the beginning of a transition to a standing posture if orthostatic intolerance conditions were previously detected.

62. The method of any of claims 42-61 and 63-70, further comprising adjusting a fall risk threshold if an orthostatic intolerance condition is detected.

63. The method of any of claims 42-62 and 64-70, further comprising prompting the device wearer to take a precautionary action if an orthostatic intolerance condition is detected.

64. The method of any of claims 42-63 and 65-70, further comprising prompting the device wearer to drink water if an orthostatic intolerance condition is detected.

65. The method of any of claims 42-64 and 66-70, further comprising providing stimulation to the device wearer when a postural transition to a standing position is detected to mitigate the effects of an orthostatic intolerance condition.

66. The method of any of claims 42-65 and 67-70, further comprising evaluating a signal from a temperature sensor while screening for an orthostatic intolerance condition.

67. The method of any of claims 42-66 and 68-70, further comprising evaluating a signal from a temperature sensor and suspend screening for the orthostatic intolerance condition if the temperature sensor indicates a temperature falling outside of a predetermined range.

68. The method of any of claims 42-67 and 69-70, further comprising processing signals from the motion sensor over a time period prior to transition of the device wearer to a standing position to characterize an activity level of the device wearer.

69. The method of any of claims 42-68 and 70, further comprising setting a threshold value for detection of an orthostatic intolerance condition based at least in part on at least one factor of the device wearer selected from age, known diagnosis, BMI, footstep speed, known gait instability, known history of orthostatic intolerance conditions, and trends thereof.

70. The method of any of claims 42-69, further comprising setting a threshold value for detection of an orthostatic intolerance condition based at least in part on known alcohol use, cardiovascular disease status, allergies, blood disorders, and prescribed medications.
